(19) United States
(12) Patent Application Publication
(10) Pub. No.: US 2025/0336008 A1
(43) Pub. Date: Oct. 30, 2025

(54) SOIL SENSOR DEVICE

(71) Applicant: Amazon Technologies, Inc., Seattle, WA (US)

(72) Inventors: Lipsy Gupta, Newark, CA (US); Jagan Vaidyanathan Rajagopalan, San Jose, CA (US); Martins Chidozie Ezuma, San Jose, CA (US); Hariharan Muthukrishnan, Milpitas, CA (US); Tzung-I Lee, San Jose, CA (US); Peruvemba Ranganath Sai Ananthanarayanan, Fremont, CA (US)

(21) Appl. No.: 18/758,597

(22) Filed: Jun. 28, 2024

Related U.S. Application Data
(60) Provisional application No. 63/640,637, filed on Apr. 30, 2024.

(51) Int. Cl. G06Q 50/02; G01J 1/42; G01K 1/024; G01N 21/25; G01N 27/22; G01N 33/24; H01Q 1/22

(52) U.S. Cl. CPC G06Q 50/02; G01J 1/4204; G01K 1/024; G01N 21/255; H01Q 1/2291; G01N 27/223; G01N 33/245; G01N 33/246

(57) ABSTRACT

Technologies directed to smart farming are described. A multimodal soil sensor device includes an elongated housing having first and second portions. An antenna is located in or above the first portion and coupled to a wireless communications component. The wireless communications component causes the antenna to radiate or receive electromagnetic energy to communicate with a second device. The multimodal soil sensor device includes one or more multimodal soil sensors to measure one or more first measurements of a first sensing modality and one or more second measurements of a second sensing modality different than the first sensing modality. The wireless communications component wirelessly sends measurement data, including the first measurements and the second measurements, to the second device via the antenna.

### RELATED APPLICATIONS

This application claims the benefit of U.S. Provisional Application No. 63/640,637, filed Apr. 30, 2024, the entire contents of which are incorporated by reference.

### BACKGROUND

Agronomy is the branch of agriculture focused on the study, management, and improvement of crops to increase their productivity and sustainability. It encompasses work on plant genetics, physiology, and soil science, as well as the integration of these disciplines to optimize plant cultivation practices. Agronomists research and apply techniques related to crop rotation, irrigation, and pest control to achieve efficient food production, while considering environmental and social impacts. Their work is crucial in addressing challenges related to food security, resource conservation, and the impact of agricultural practices on the environment.

### BRIEF DESCRIPTION OF DRAWINGS

The present inventions will be understood more fully from the detailed description given below and from the accompanying drawings of various embodiments of the present invention, which, however, should not be taken to limit the present invention to the specific embodiments, but are for explanation and understanding only.

FIG. 1 is a block diagram of a wireless multimodal soil sensor device according to at least one embodiment.

FIG. 2A-FIG. 2B are block diagrams of multimodal soil sensor device with dual-purpose antennas according to at least one embodiment.

FIG. 3A is a perspective view of an external bottom housing of a wireless multimodal soil sensor device according to at least one embodiment.

FIG. 3B is a side view of the external bottom housing of FIG. 3A according to at least one embodiment.

FIG. 4A is a side view of an inner carrier of an external bottom housing according to at least one embodiment.

FIG. 4B is a side view of an outer casing of the external bottom housing of FIG. 4A according to at least one embodiment.

FIG. 5 is a perspective view of a multimodal soil sensor device according to at least one embodiment.

FIG. 6 is a side view of a multimodal soil sensor device with an external antenna coupled by an RF cable according to at least one embodiment.

FIG. 7 is a schematic diagram of a multimodal soil sensor device according to at least one embodiment.

### DETAILED DESCRIPTION

The present application discloses one or more preferred implementations of a wireless sensor device, e.g. a wireless sensor device for use with smart farming approaches.

Technologies related to smart farming are described. Smart Farming may, for example, refer to use of Internet of Things (IoT) sensors, location services, robotics, artificial intelligence/machine learning (AI/ML) technologies to improve yield, quality of produce while reducing cost of cultivation. Smart farming can be used to monitor key crop growth parameters and generate precise agronomy advice for soil nutrients (e.g., Nitrogen-Phosphorus-Potassium (NPK) content), fertigation, irrigation, Growing degree Days (GDD), Pest and Disease detection, and the like.

Current solutions have various entry barriers to adopt smart farming, including cost of connectivity, cost of sensors, precision, and easy access to agronomy. In particular, current solutions lack affordable, reliable and easy to deploy connectivity since one wireless moisture sensors costs multiple hundreds of dollars and a gateway for connectivity costs even more. Currently, there are no accurate or precise in-situ field sensors. Instead, farmers rely on infrequent soil lab testing and macro-level information from satellite imagery and weather stations. Also, many farmers do not have easy access to agronomy and precise cultivation practices.

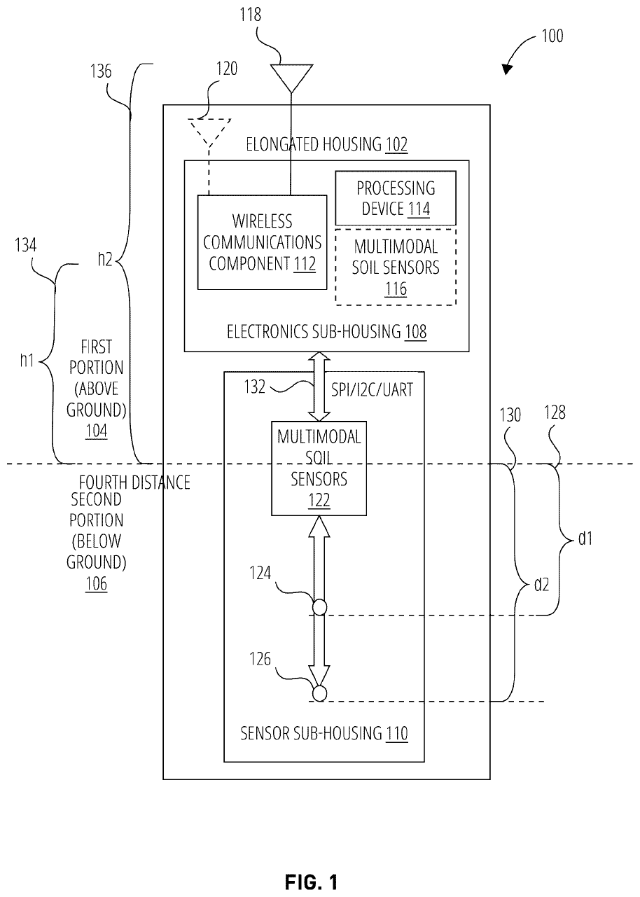

Aspects and embodiments of the present disclosure utilize low-cost, in-situ, wireless multimodal soil sensor devices. Data from these soil sensor devices can be used to predict soil nutrients, GDD, irrigation conditions, and pests and disease conditions. The wireless multimodal soil sensor devices can be referred to herein as “wireless probe devices,” “soil sensors,” “wireless sensors,” “sensors,” “probe devices,” “soil probes,” “end node,” “IoT device,” or “sensor end node.” Additional details of the wireless multimodal soil sensor devices are described below with respect to FIG. 1 to FIG. 7.

FIG. 1 is a block diagram of a wireless multimodal soil sensor device 100 according to at least one embodiment. The wireless multimodal soil sensor device 100 includes an elongated housing 102 having a first portion 104 and a second portion 106. The first portion 104 can be above ground when the wireless multimodal soil sensor device 100 is placed in the ground (e.g., placed in the soil). The second portion 106 can be below ground when the wireless multimodal soil sensor device 100 is placed in the ground. The second portion 106 can be a stem or have a stem shape that can be placed in the soil with the first portion 104 remaining above ground, much like a flower bulb on top of the stem. Within the elongated housing 102, the wireless multimodal soil sensor device 100 includes electronics sub-housing 108 and sensor sub-housing 110.

The wireless multimodal soil sensor device 100 can include a wireless communications component 112, a processing device 114, and optional multimodal soil sensors 116 located within the electronics sub-housing 108. The wireless communications component 112 is coupled to an external antenna 118, an internal antenna 120, or both. The antenna 118 and the antenna 120 can be located in or above the first portion 104. The wireless communications component 112 is located in the first portion 104. The wireless communications component 112 can cause the antenna 118 (or antenna 120) to radiate or receive electromagnetic energy to communicate data with a second device (not illustrated in FIG. 1).

The processing device 114 can be an electronic component or system designed to execute programmed instructions for the purpose of data processing. This device may encompass a wide variety of hardware such as microprocessors, microcontrollers, digital signal processors (DSPs), application-specific integrated circuits (ASICs), field-programmable gate arrays (FPGAs), or other integrated or discrete logic circuits. The processing device 114 can perform tasks such as arithmetic and logic operations, controlling digital systems, processing input data from external devices, and managing data output for various applications. The processing device 114 operates based on a set of instructions, which could be part of software applications, firmware, or embedded code, tailored to specific tasks or general-purpose agronomy engine or an agronomy application. The instructions, when executed by the processing device 114, cause the processing device 114 to identify at least one farming action to be performed based on the measurement data. In at least one embodiment, the agronomy engine can host one or more AI/ML models trained to predict localized farm parameters of at least a portion of a farm. The localized farm parameters can include soil nutrients, GDD, irrigation conditions, pest and disease conditions, or the like. In other embodiments, the agronomy engine can implement other threshold-based formulas to predict or otherwise determine localized farm parameters. In some embodiments, historical localized farm parameters can be used to predict current localized farm parameters. In other embodiments, historical farm parameters from other farms can be used to predict current localized farm parameters of a farm.

The processing device 114 is coupled to the wireless communications component 112. The processing device 114 can control the wireless communications component 112 to communicate data with the second device. The processing device 114 can aggregate and process the collected data before sending to the second device using the wireless communications component 112. In some embodiments, as described in more detail below, the processing device 114 can host one or more trained AI/ML models or applications to predict soil nutrients, GDD, irrigation conditions, pests and disease conditions, or the like.

In at least one embodiment, the wireless communications component 112 is coupled to the antenna 118 using an RF connector, such as SubMiniature version A (SMA) connector. SMA connector is a type of RF connector used for connecting radio frequency coaxial cables. SMA connectors are used in radio communications because of their small size and excellent electrical performance. They are characterized by their screw-type coupling mechanism, which provides a high degree of mechanical stability and minimizes losses at high frequencies. SMA connectors are widely used in both commercial and consumer communications devices to facilitate reliable high-frequency connections. In at least one embodiment, the antenna 118 (or antenna 120) is a hybrid antenna that can be used for different radio technologies, such as the Long Range Wide Area Network (LoRaWAN) protocol (or other chirp spread spectrum protocol or modulation scheme) and the Bluetooth Low Energy (BLE) protocol. That is, the antenna 118 can be a hybrid LoRa+BLE antenna. As described above, the antenna 118 can be an external antenna that is coupled to the wireless communications component 112 via an RF connector. The antenna 118 can have a flexible Cushcraft type antenna structure to add antenna height. Cushcraft refers to a brand known for designing and manufacturing a wide range of antennas for amateur radio, commercial, and military applications. Although illustrated as being external to the electronics sub-housing 108, the antenna 118 can be located within the sensor sub-housing 110, such as illustrated with antenna 120.

In at least one embodiment, the wireless communications component 112 can communicate the measurement data using the LoRaWAN protocol or the BLE protocol. In at least one embodiment, the wireless communications component 112 can include a hybrid LoRaWAN+BLE module that combines the long-range, low-power communication capabilities of the LoRaWAN protocol with the short-range, high-throughput abilities of BLE protocol. This hybrid module enables devices to communicate over vast distances using the LoRaWAN protocol while also facilitating close-proximity interactions via the BLE protocol. More specifically, the LoRaWAN protocol is designed for long-range communications, enabling devices to transmit data over distances of up to several kilometers in rural areas and several hundred meters in urban environments, making it ideal for IoT applications that require wide coverage and minimal power consumption, such as environmental monitoring or smart agriculture. The BLE protocol focuses on short-range communication, typically effective within tens of meters, and is designed to provide high data rates with very low power consumption. The BLE protocol is widely used for wearable devices, fitness trackers, and wireless peripherals, offering efficient connectivity and easy pairing with smartphones and tablets. The integration of LoRaWAN and BLE protocols in a single module allows for versatile IoT devices capable of leveraging the broad coverage of LoRaWAN for remote data transmission and the convenience and bandwidth of BLE for local, high-speed data exchange. This combination supports a wide range of innovative applications, including smart cities, industrial IoT, and connected healthcare, where devices can benefit from both extensive reach and the ability to interact with users' smartphones and other BLE-equipped devices.

In at least one embodiment, the wireless communications component 112 and the processing device 114 are separate components, as illustrated in FIG. 1. In at least one embodiment, the wireless communications component 112 and the processing device 114 are integrated into a single radio and controller unit, such as a wireless module microcontroller unit (MCU) that supports protocol operations (e.g., a hybrid LoRaWAN and BLE module), and optionally some sensor operations. For example, the wireless module MCU can support operations for a temperature sensor, a relative humidity (Rh) sensor, and other controller operations, as described herein.

In at least one embodiment, the multimodal soil sensors 122 can collect field data using different modalities to obtain information about the soil, moisture, temperature, relative humidity, and other environmental conditions at a location of the wireless multimodal soil sensor device 100. Each of the multimodal soil sensors 122 can include circuitry to measure an attribute of a sensor probe (also referred to as a contact point, contact area, or electrode). The multimodal soil sensors 122 can use one or more sensor probes at varying distances from the first portion 104, in the sensor sub-housing 110, the varying distances corresponding to varying depths in the soil. For example, as illustrated in FIG. 1, the multimodal soil sensors 122 can use a first sensor probe 124, located at a first distance 128 (e.g., 6 inches) from the first portion 104, and a second sensor probe 126, located at a second distance 130 (e.g., 10 inches). In other embodiments, the multimodal soil sensors 122 can use sensor probes at various distances, corresponding to various depths, such as 8 inches, 12 inches, 18 inches for various crop health monitoring. The second distance 130 is greater than the first distance 128. The multimodal soil sensors 122 can includes capacitive moisture sensors with the electrodes placed at different distances from the first portion 104. The multimodal soil sensors 122 is coupled to electronics in the electronics sub-housing 108 using an interconnect 132. The sensor probes at the varying distances can be used to obtain soil parameter measurements at different depths in the soil. In at least one embodiment, the wireless communications component 112 can be located at a third distance 134 from the second portion 106, corresponding to a first height (h1) above the soil, and the antenna 118 can be located at a fourth distance 136 from the second portion 106, corresponding to a second height (h2) above the soil. For some crops, the antenna 118 and/or the wireless communications component 112 need to be located at higher positions. A wireless multimodal soil sensor device 100 with an elongated stem design can be used for those crops. Alternatively, the wireless multimodal soil sensor device 100 can have a telescoping mechanism to position the antenna 118 (and/or the wireless communications component 112) at various heights above the soil.

In at least one embodiment, the multimodal soil sensors 122 can measure Electrical Conductivity (EC), Nitrogen, Phosphorus, Potassium (NPK), potential of hydrogen (pH) levels, soil moisture levels below ground. The EC, NPK, pH, and soil moisture measurements are key parameters for assessing soil health and fertility. EC (Electrical Conductivity) measures the soil's ability to conduct electricity, which is directly related to the concentration of soluble salts in the soil. It is an indicator of the soil's salinity that can affect plant growth. NPK (Nitrogen, Phosphorus, Potassium) represents the three primary nutrients required by plants. Nitrogen is essential for leaf growth, phosphorus for root and flower development, and potassium for overall health and disease resistance. pH indicates the acidity or alkalinity of the soil on a scale from 0 to 14, where 7 is neutral. Soil pH affects nutrient availability to plants and can significantly influence plant growth. Soil moisture refers to the amount of water present in the soil accessible to plants. Soil moisture is needed for seed germination, nutrient solubility, and the overall physiological functions of plants. Together, these factors can be used for determining the suitability of soil for specific crops and guiding agricultural practices to optimize crop health and yield. In at least one embodiment, the multimodal soil sensors 122 (and/or the optional multimodal soil sensors 116) include a temperature sensor, a relative humidity (RH) sensor, a light sensor (also referred to as LUX sensor) to measure three parameters: temperature, humidity, and light intensity. The temperature sensor is a component that measures the ambient temperature of the environment. It can be based on various technologies, including thermistors, resistance temperature detectors (RTDs), or semiconductor-based sensors. Temperature sensors are crucial for applications requiring precise climate control, such as agriculture, manufacturing, and smart home systems. The RH sensors measure the amount of water vapor in the air relative to the maximum amount of water vapor the air can hold at a given temperature. Expressed as a percentage, RH is a critical factor in environmental monitoring. A LUX sensor measures illuminance, which is a metric for the perceived intensity of light as detected by the human eye. The unit of measurement is Lux, representing lumens per square meter. LUX sensors are used to monitor and control lighting conditions in various settings. The LUX sensors can be used in this agricultural technology to ensure plants receive the optimal light levels for growth. Combining these sensors into a single device or system enables detailed monitoring and management of environmental conditions. This can facilitate automated adjustments in smart systems to maintain optimal conditions for specific farming requirements. In at least one embodiment, the wireless multimodal soil sensor device 100 can obtain first measurements of a first sensing modality and second measurements of a second sensing modality different than the first sensing modality. The first measurements and the second measurements can be at least two of the following sensing modalities: temperature measurements, EC measurements, NPK content measurements, soil moisture measurements, a pH measurements, Rh measurements, and illuminance measurements. In other embodiments, the wireless multimodal soil sensor device 100 can obtain other measurements of other sensing modalities.

As described above, the second portion 106 can be a stem or have a stem shape and the multimodal soil sensors 122 can be integrated into the stem that is placed in the soil. The multimodal soil sensors 122 can communicate measurement data to the processing device 114 or wireless communications component 112 over the interconnect 132. The sensor sub-housing 110 can be a cylindrical housing with a collar as a ground level indicator. In at least one embodiment, some of the multimodal soil sensors 122 can be integrated into a main body of the sensor sub-housing 110 below the collar for in-ground measurements, and some of the multimodal soil sensors 122 can be integrated into a neck of the sensor sub-housing 110 for above-ground measurements. These soil sensors can be in addition to, or substitutes of, the multimodal soil sensors 116 in the electronics sub-housing 108.

The multimodal soil sensors 122 can measure one or more measurements and send the one or more measurements to the processing device 114 over the interconnect 132 using various interconnect protocols, such as Serial Peripheral Interface (SPI), Universal Asynchronous Receiver/Transceiver (UART), Inter-Integrated Circuit (I2C) protocols. The SPI, I2C, and UART protocols are communication protocols widely used in electronic systems for interfacing microcontrollers with various peripherals, sensors, and other microcontrollers. SPI is a synchronous serial communication protocol known for its high-speed data transfer. It operates on a primary-secondary architecture (principal-follower architecture), where the primary device controls the communication with one or more secondary devices. Key features include separate data lines for sending and receiving data (MOSI and MISO), a clock line (SCK), and a chip select line for each secondary device. I2C, also a synchronous communication protocol, uses only two wires for communication, making it ideal for connecting multiple secondary devices to a single primary device, thereby reducing the complexity of wiring. These two wires are the serial data line (SDA) and the serial clock line (SCL). I2C supports multiple primary and secondary devices on the same bus, with hardware addressing used to communicate with specific devices. UART is an asynchronous serial communication protocol that does not use a clock signal to synchronize the transmission. Instead, data is sent in packets framed by start and stop bits at a pre-agreed baud rate. UART is simple to use and highly versatile, making it suitable for communication between a microcontroller and peripheral devices or between two microcontrollers. Each of these protocols has its advantages and is chosen based on the specific requirements of the application, including speed, data volume, complexity, and resource constraints of the system.

In at least one embodiment, the multimodal soil sensors 122 can measure one or more first measurements of a first sensing modality and one or more second measurements of a second sensing modality different than the first sensing modality. In some cases, the modalities can be more than two modalities, such as four different modalities. The measurement data, including at least the first measurements and the second measurements collected by the multimodal soil sensors 122, can be sent to the processing device 114 to be sent wirelessly to the second device via the wireless communications component 112 and the antenna 118 (or antenna 120).

In at least one embodiment, the wireless multimodal soil sensor device 100 is a single integrated housing that can be put in the ground, leaving the first portion 104 above ground and the second portion 106 below ground. In at least one embodiment, the wireless multimodal soil sensor device 100 is two separate components that are secured together using mechanical couplings. For example, the electronics sub-housing 108 can have first threads that mate with second threads of the sensor sub-housing 110. That is, a bottom portion of the electronics sub-housing 108 can be screwed onto a threaded region at a top end of the sensor sub-housing 110. In at least one embodiment, the wireless multimodal soil sensor device 100 includes i) a first temperature sensor, a first relative humidity (Rh) sensor, and an illuminance sensor located in the first portion 104, ii) a second temperature sensor and a first electrical conductivity (EC) sensor located in the second portion 106 at the first distance 128 from a bottom of the elongated housing, and iii) a third temperature sensor, a second EC sensor, and a pH sensor located in the second portion 106 at the second distance 130 from the bottom of the elongated housing.

In a further embodiment, the wireless multimodal soil sensor device 100 includes a first capacitive moisture sensor located in the second portion 106 between the first distance 128 and the first portion 104, and a second capacitive moisture sensor located in the second portion 106 between the first distance 128 and the second distance 130.

In at least one embodiment, the elongated housing 102 of the wireless multimodal soil sensor device 100 includes a probe housing having first threads at a first end and an electronics housing having second threads to mechanically couple with the first threads. The wireless communications component is located within the electronics housing. The probe housing includes a stem shape to be placed in soil at a second end opposite the first end. One or more multimodal soil sensors are placed in the probe housing.

In at least one embodiment, the electronics sub-housing 108 includes a memory device operatively coupled to the processing device 114. The memory component can be a component or subsystem within the wireless multimodal soil sensor device 100 used to store data or instructions for processing. The memory device can retain the operational information and processed data of wireless multimodal soil sensor device 100. The memory device can be volatile or non-volatile memory. The memory device can store the measurement data and instructions of an agronomy engine (or an agronomy application). The instructions of the agronomy engine, when executed by the processing device 114, cause the processing device 114 to identify at least one farming action to be performed based on the measurement data. In at least one embodiment, the agronomy engine can host one or more AI/ML models trained to predict localized farm parameters of at least a portion of a farm. In at least one embodiment, the agronomy engine can use one or more threshold-based processes or algorithms to predict or determine localized farm parameters of at least a portion of a farm (or indoor house plants, lawns, gardens, etc.). The localized farm parameters can include soil nutrients, GDD, irrigation conditions, pest and disease conditions, or the like.

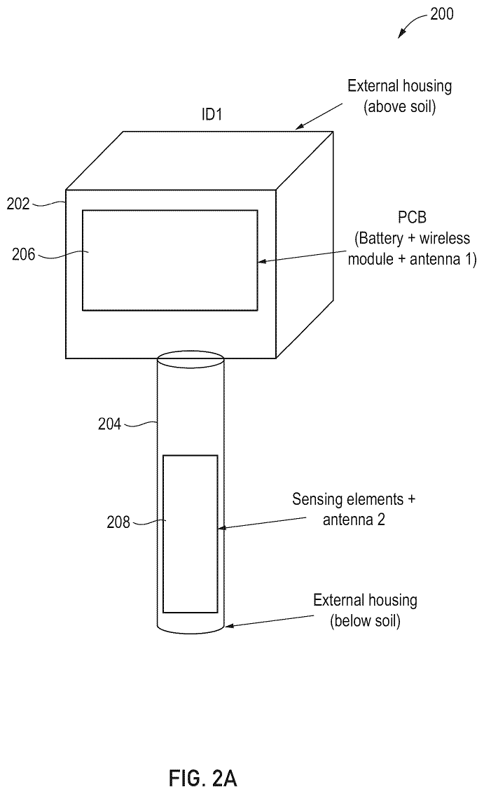

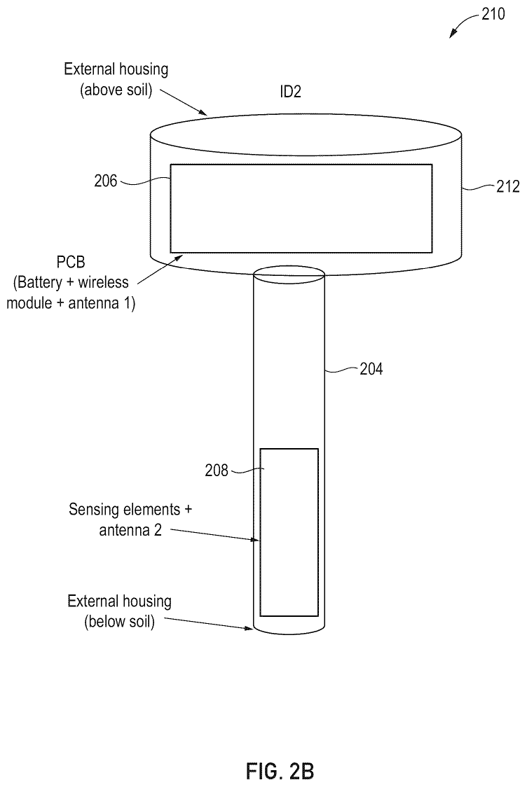

FIG. 2A-FIG. 2B are block diagrams of multimodal soil sensor device with dual-purpose antennas according to at least one embodiment. The multimodal soil sensor device 200 of FIG. 2A includes an external top housing 202 and an external bottom housing 204. When the multimodal soil sensor device 200 is placed in the soil, the external top housing 202 is located above the soil, and the external bottom housing 204 is located below the soil. The multimodal soil sensor device 200 includes a printed circuit board (PCB) 206 with a first antenna and some of the electronics, such as the batter, the wireless module, a temperature sensor, a light sensor, or the like. The PCB with first antenna 206 can be located in the external top housing 202. The multimodal soil sensor device 200 include sensing elements with a second antenna 208. The sensing elements with second antenna 208 can be located in the external bottom housing 204.

The multimodal soil sensor device 210 of FIG. 2B is similar to multimodal soil sensor device 200 as noted by similar reference numbers, except the multimodal soil sensor device 210 includes an external housing 212 that has a different shape than the external top housing 202. As similar to multimodal soil sensor device 200, the multimodal soil sensor device 210 includes PCB with first antenna 206 and sensing elements with second antenna 208.

As illustrated in FIG. 2A and FIG. 2B, the PCB with first antenna 206 can use the external top housing 202 (or external housing 212) as a main resonant arm and the external bottom housing 204, below the soil, as a ground leg to radiate electromagnetic energy to communicate measurement data with another device. This can help in provide a smaller antenna size with the ability to integrate in the small form factor and provide better antenna radiation as the ground leg is buried under the soil. FIG. 2A and FIG. 2B show two different industrial designs (ID 1 and ID 2). In both ID 1 and ID 2, there is a portion of an external top housing 202 (or external housing 212) (above soil) with the PCB. As described above, the PCB can include the battery, the wireless module and the first antenna (antenna 1). The PCB can also include some of the sensors described herein to obtain measurements above the soil. In both IDS 1 and ID 2, there is another portion of the external housing, namely the external bottom housing 204 (below soil) with the sensing elements and the second antenna (antenna 2). The first and second antennas (or respective antenna elements) can be used for radiating electromagnetic energy to communicate measurement data with another device. The first and second antennas (or respective antenna elements) can be used for sensing water flow as described below.

As illustrated in FIGS. 2A and 2B, the first antenna 1 is placed above ground and the second antenna 2 is placed inside the soil. Using a change in impedance and isolation between the two antennas, the first and second antennas can be used to identify whether water is flowing. A processing device disposed on the PCB can measure a change in impedance and isolation between the first antenna 1 and the second antenna 2 and determine whether water is flowing.

In some embodiments, the PCB in the external top housing 202 (or external housing 212) above ground can have a light sensing element (like ambient light sensor, spectroradiometer, etc.). Using the light sensor, the multimodal soil sensor device 200 (or multimodal soil sensor device 210) can measure a greenness level of the vegetation above the ground level and calculate the Normalized Difference Vegetation Index (NDVI) of the plant.

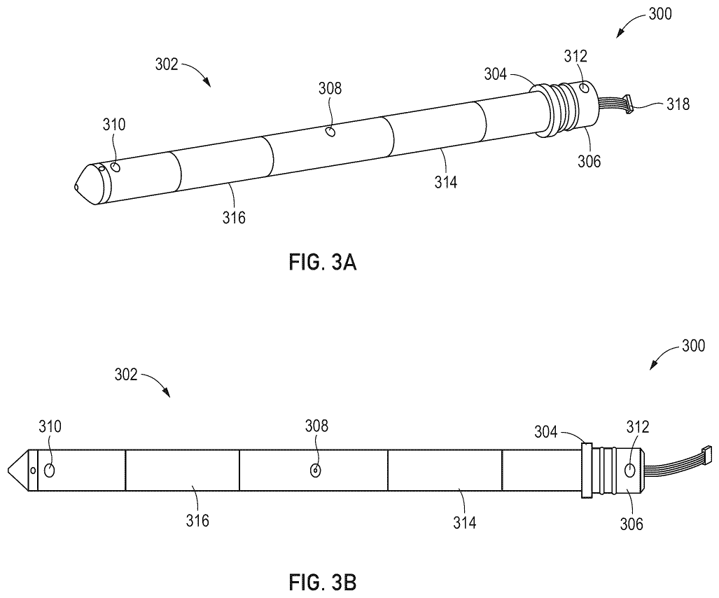

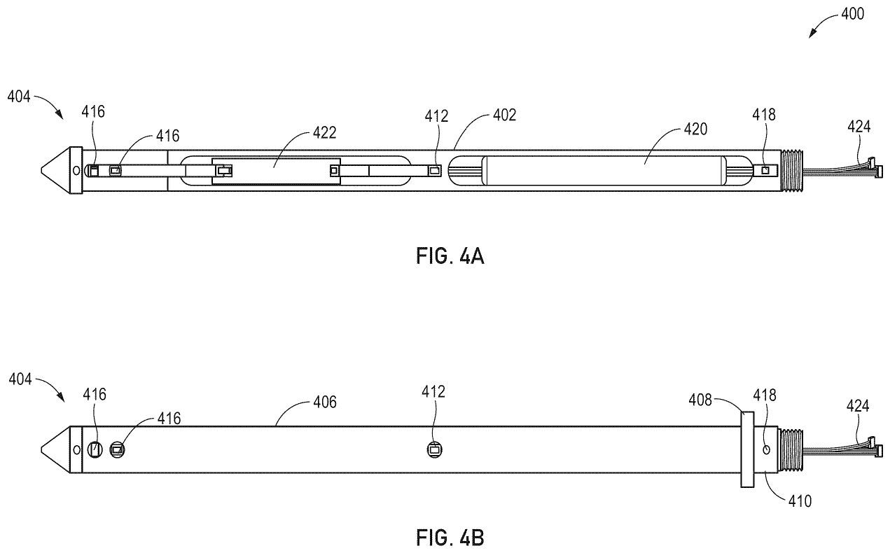

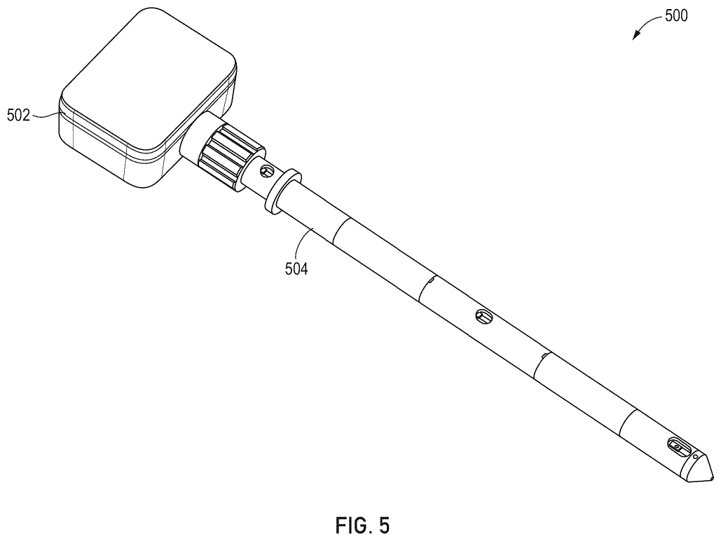

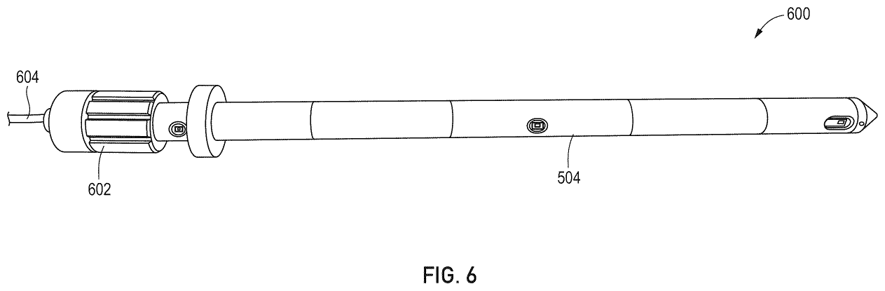

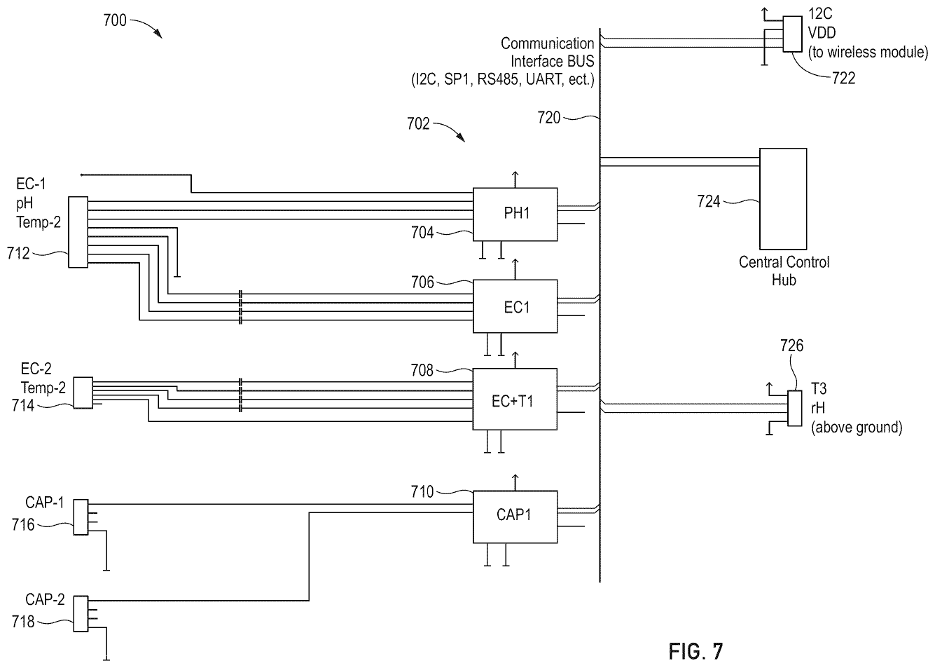

Examples of the external bottom housing 204 are illustrated in FIG. 3A, FIG. 3B, FIG. 4A, FIG. 4B, FIG. 5, and FIG. 6. Examples of the external top housing 202 or external housing 212 are illustrated in FIG. 5 and FIG. 6.

FIG. 3A is a perspective view of an external bottom housing 300 of a wireless multimodal soil sensor device according to at least one embodiment. The external bottom housing 300 can be the second portion 106 of FIG. 1. As described above with respect to the second portion 106, the external bottom housing 300 can be a stem or have a stem shape where the multimodal soil sensors are integrated into the stem that is placed in the soil. The external bottom housing 300 can be a cylindrical housing with a main body 302, a collar 304, and a neck 306, the collar 304 representing a ground level indicator. In at least one embodiment, some of the multimodal soil sensors 122 can be integrated into a main body 302 below the collar 304 for in-ground measurements, and some of the multimodal soil sensors 122 can be integrated into the neck 306 for above-ground measurements.

As described above, the external bottom housing 300 includes the main body 302, the collar 304, and the neck 306. One or more sensor probes can be disposed in the main body 302 or the neck 306. As illustrated, for example, the external bottom housing 300 includes a first sensor probe 308 located at a first distance from the collar 304, and a second sensor probe 310 located at a second distance from the collar 304, the second distance being greater than the first distance. A third sensor probe 312 is located in the neck 306 above the collar 304. It should be noted that sensor probe 312 can be located above ground and does not have to be close to the soil surface. The sensor probe 312 can be at a height depending on a length of the stem for the radio module. As described herein, one or more capacitive moisture sensors can be disposed in the main body 302, where the main body 302 is placed in the soil. As illustrated, for example, the external bottom housing 300 include a first capacitive moisture sensor 314 located between the first distance at the collar 304, and a second capacitive moisture sensor 316 located between the first distance and the second distance.

In at least one embodiment, the first sensor probe 308 includes an EC sensor and a temperature sensor, such as illustrated in FIG. 3B. In at least one embodiment, the second sensor probe 310 includes an EC sensor, a pH sensor, and a temperature sensor, such as illustrated in FIG. 3B. In at least one embodiment, the third sensor probe 312 includes a relative humidity (Rh) sensor and a temperature sensor, such as illustrated in FIG. 3B. Alternatively, the external bottom housing 300 can include more or less sensor probes than illustrated in FIG. 3A-FIG. 3B. Also, different sensing modalities can be implemented at the different sensor probes in the external bottom housing 300.

In a further embodiment, the wireless multimodal soil sensor device 100 includes a first capacitive moisture sensor located in the second portion 106 between the first distance 128 and the first portion 104, and a second capacitive moisture sensor located in the second portion 106 between the first distance 128 and the second distance 130. Alternatively, the external bottom housing 300 can include more or less capacitive moisture sensors than illustrated in FIG. 3A.

The sensor probes 308-312 can communicate measurement data to a processing device (e.g., processing device 114 or a wireless communications component (e.g., wireless communications component 112 of FIG. 1) over an interconnect 318, similar to the interconnect 132 described above with respect to FIG. 1. In this embodiment, the interconnect 318 includes a flexible cable that connects the probe sensors sensor probes 308, 310, 312, and the first capacitive moisture sensors 314 and 316 to electronics in the first portion 104 (e.g., first portion 104 of elongated housing 102 or external top housing 202 of FIG. 2A and FIG. 2B).

FIG. 3B is a side view of the external bottom housing 300 of FIG. 3A according to at least one embodiment. As illustrated in FIG. 3B, the external bottom housing 300 can have specific dimensions and the one or more probe sensors can be located at specific lengths, corresponding to specific depths in the soil. Although FIG. 3B identifies some example dimensions, the external bottom housing 300 can have varying dimensions. For example, the first sensor probe 308 is located at 6 inches from the collar 304 and the second sensor probe 310 is located at 12 inches from the collar 304. Alternatively, the sensor probes can be located at different distances.

As illustrated in FIG. 3B, the neck 306 can include threads and one or more O-rings to couple the external bottom housing 300 to an external top housing (e.g., first portion 104 of elongated housing 102 or external top housing 202 of FIG. 2A and FIG. 2B).

FIG. 4A is a side view of an inner carrier 402 of an external bottom housing 400 according to at least one embodiment. The external bottom housing 400 can be the second portion 106 of FIG. 1. As described above with respect to the second portion 106, the external bottom housing 400 can be a stem or have a stem shape where the multimodal soil sensors are integrated into the stem that is placed in the soil. The external bottom housing 400 can have an inner carrier 402 (illustrated in FIG. 4A) and an outer casing 406 (illustrated in FIG. 4B). The external bottom housing 400 can be a cylindrical housing with a main body 404, a collar 408, and a neck 410, the collar 408 representing a ground level indicator. In at least one embodiment, some of the multimodal soil sensors 122 can be integrated into the inner carrier 402 below the collar 408 for in-ground measurements, and some of the multimodal soil sensors 122 can be integrated into the neck 410 for above-ground measurements.

As described above, the external bottom housing 400 includes the main body 404, the collar 408, and the neck 410. One or more sensor probes can be disposed in the main body 404 or the neck 410. As illustrated, for example, the inner carrier 402 includes a first sensor probe 412 located at a first distance from the collar 408, a second sensor probe 414 located at a second distance from the collar 408, the second distance being greater than the first distance, and a third sensor probe 416 located at a third distance from the collar 408, the third distance being greater than the second distance. A fourth sensor probe 418 is located in the neck 410 above the collar 408, when the outer casing 406 is placed over the inner carrier 402. The fourth sensor probe 418 can be located above ground and does not have to be close to the soil surface. The sensor probe 312 can be at a height depending on a length of the stem for the radio module. In another embodiment, one or more capacitive moisture sensors can be disposed in the main body 302.

In at least one embodiment, the first sensor probe 412 includes an EC sensor and a temperature sensor, such as illustrated in FIG. 4B. In at least one embodiment, the second sensor probe 414 includes an EC sensor and a temperature sensor, such as illustrated in FIG. 4B. In at least one embodiment, the third sensor probe 416 includes a pH sensor, such as illustrated in FIG. 4B. In at least one embodiment, the fourth sensor probe 418 includes a relative humidity (Rh) sensor and a temperature sensor, such as illustrated in FIG. 4B. Alternatively, the external bottom housing 400 can include more or less sensor probes than illustrated in FIG. 4A-FIG. 4B. Also, different sensing modalities can be implemented at the different sensor probes in the external bottom housing 400.

The sensor probes 412-418 can communicate measurement data to a processing device (e.g., processing device 114 or a wireless communications component (e.g., wireless communications component 112 of FIG. 1) over an interconnect 424, similar to the interconnect 132 described above with respect to FIG. 1. In this embodiment, the interconnect 424 includes two flexible cables that that connect the probe sensors sensor probes 412, 414, 416, and 418 to electronics in the first portion 104 (e.g., first portion 104 of elongated housing 102 or external top housing 202 of FIG. 2A and FIG. 2B).

As illustrated in FIG. 4A, the external bottom housing 400 includes a battery compartment to secure a battery. In at least one embodiment, the batter is coupled to the electronics via one of the flexible cables, such as to a power management sub-system on the main circuit board in the external top housing. In at least one embodiment, the external bottom housing 400 includes a sensor interface board 422. The sensor interface board 422 can include circuitry of the multimodal soil sensors that are connected to the sensor probes 412-418. The sensor interface board 422 can be coupled to the electronics via one of the flexible cables.

In at least one embodiment, the external bottom housing 400 is part of an electronic device. The electronic device can have an external housing with a first portion and a second portion. The first portion is disposed proximate a first end of the electronic device, and the second portion is disposed further from the first end than the first portion. The external bottom housing 400 is the second portion. In at least one embodiment, the battery 420 can be disposed at least partially within the inner carrier 402. Alternatively, the battery 420 can be disposed at least partially within the first portion. In at least one embodiment, the electronic device includes a first antenna and/or a wireless communications component disposed at least partially within the first portion. A second antenna can be disposed at least partially within the external bottom housing 400.

FIG. 4B is a side view of an outer casing 406 of the external bottom housing of FIG. 4A according to at least one embodiment. As illustrated in FIG. 4B, the outer casing 406 can cover the battery 420, the sensor interface board 422 and other circuitry within the inner carrier 402, where openings in the outer casing 406 can expose the probe sensors. The openings can have glass or plastic covering to protect the probe sensors from soil, water, or the like. As illustrated in FIG. 4B, the external bottom housing 400 can have specific dimensions and the one or more probe sensors can be located at specific lengths, corresponding to specific depths in the soil. Although FIG. 4B identifies some example dimensions, the external bottom housing 400 can have varying dimensions. For example, the first sensor probe 412 is located at 6 inches from the collar 408 and the second sensor probe 414 is located at approximately 10 inches from the collar 408. Alternatively, the sensor probes can be located at different distances.

As illustrated in FIG. 4A and FIG. 4B, the inner carrier 402 can include threads to couple the external bottom housing 400 to an external top housing (e.g., first portion 104 of elongated housing 102 or external top housing 202 of FIG. 2A and FIG. 2B).

FIG. 5 is a perspective view of a multimodal soil sensor device 500 according to at least one embodiment. The multimodal soil sensor device 500 is a prototype with a wireless module 502 secured to a top end of a stem 504 with one or more multimodal soil sensors. The wireless module 502 is a portion of the multimodal soil sensor device 500 that is located above soil when the multimodal soil sensor device 500 is placed in the soil. The stem 504 is the portion of the multimodal soil sensor device 500 that is located in the soil when the multimodal soil sensor device 500 is placed in the soil. The stem 504 can include different modality sensors at different depths of the stem 504, which is placed in the ground. In this embodiment, the wireless module 502 has an electronics housing that is secured to the stem 504 using mating threads. The stem 504 can include a collar as a ground level indicator. Also, as illustrated in FIG. 5, the multimodal soil sensor device 500 can include multiple capacitive moisture sensors.

FIG. 6 is a side view of a multimodal soil sensor device 600 with an external antenna coupled by an RF cable 604 according to at least one embodiment. The multimodal soil sensor device 600 is similar to the multimodal soil sensor device 500 as noted by similar reference numbers, except the multimodal soil sensor device 600 includes a wireless module 602. The wireless module 602 includes a wireless communications component contained within a housing and the RF cable 604 connects the wireless communications component to an external antenna (not illustrated in FIG. 6). The RF cable 604 can connect to an external antenna that adds height to the multimodal soil sensor device 600 for communications with another device. As indicated above, the multimodal soil sensor device 600 can include a collar as a ground level indicator. The RF cable or other types of RF connectors can be used to extend the height of the antenna above the ground.

FIG. 7 is a schematic diagram of multimodal soil sensors 702 of a multimodal soil sensor device 700 according to at least one embodiment. The multimodal soil sensors 702 can include a pH sensor 704, an EC sensor 706, an EC and temperature sensor 708, and a capacitive sensor 710. The pH sensor 704 and EC and temperature sensor 706 are coupled to a first connector 712. The first connector 712 can be coupled to one or more sensor probes, such as those illustrated and described above with respect to FIG. 3A to FIG. 7. The one or more probes can be located in openings in the housing at different lengths of the multimodal soil sensor device 700. The EC and temperature sensor 708 is coupled to a second connector 714. The second connector 714 can be coupled to one or more sensor probes located in openings in the housing of the multimodal soil sensor device 700. These sensor probes can be located at a different length than the sensor probes coupled to the first connector 712. The capacitive sensor 710 is coupled to a third connector 716 and a fourth connector 718. The third connector 716 is coupled to a first capacitive moisture sensor. The fourth connector 718 is coupled to a second capacitive moisture sensor. The capacitive moisture sensors can be located at different lengths of the multimodal soil sensor device 700.

As illustrated in FIG. 7, the pH sensor 704, the EC and temperature sensor 706, the EC and temperature sensor 708, and the capacitive sensor 710 ac coupled to a bus 720 (e.g., I2C bus). The pH sensor 704, EC and temperature sensor 706, EC and temperature sensor 708, and capacitive sensor 710 can send measurement data to other components via a fifth connector 722. The fifth connector 722 can be coupled to the processing device, the wireless communications component, the LoRaWAN module, or other components as described herein. A general-purpose input-output (GPIO) expander 724 can also be coupled to the bus 720. The GPIO expander 724 can expand connections to the components coupled to the bus 720. In at least one embodiment, a third temperature sensor and a RH sensor can be coupled to the bus 720 via a sixth connector 726. The third temperature sensor and the RH sensor can be located in the first portion (above ground) of the multimodal soil sensor device 700, whereas the temperature sensors of the EC and temperature sensor 706 and EC and temperature sensor 708 are located in the second portion (below ground) of the multimodal soil sensor device 700. For example, the third temperature sensor and the RH sensor, which are coupled to the sixth connector 726, can be located in the neck above ground level.

In at least one embodiment, the fifth connector 722 can be a 4-pole header (2.54 mm pitch) in which an I2C, 3.3V supply line comes out. For a mechanical fit, a thread can be made at the head. This screws the external bottom housing of the multimodal soil sensor device 700 to the external top housing (e.g., housing the LoRaWAN module). The multimodal soil sensors 702 can output raw values. In some embodiments, the multimodal soil sensors 702 can perform some processing of the raw data and output processed data. In at least one embodiment, post processing of the raw data (or processed data) can occur in the components coupled to the fifth connector 722, such as the LoRaWAN module, or the like. In other embodiments, the LoRaWAN module can send the raw data to a computing system, such as a cloud computing system, a laptop, a tablet, a phone, a personal computer, or the like.

In the above description, numerous details are set forth. It will be apparent, however, to one of ordinary skill in the art having the benefit of this disclosure, that embodiments may be practiced without these specific details. In some instances, well-known structures and devices are shown in block diagram form rather than in detail in order to avoid obscuring the description.

Some portions of the detailed description are presented in terms of algorithms and symbolic representations of operations on data bits within a computer memory. These algorithmic descriptions and representations are the means used by those skilled in the data processing arts to convey the substance of their work most effectively to others skilled in the art. An algorithm is used herein and is generally conceived to be a self-consistent sequence of steps leading to the desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “determining,” “sending,” “receiving,” “scheduling,” or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

Embodiments also relate to an apparatus for performing the operations herein. This apparatus may be specially constructed for the required purposes, or it may comprise a general-purpose computer selectively activated or reconfigured by a computer program stored in the computer. Such a computer program may be stored in a computer-readable storage medium, such as, but not limited to, any type of disk including floppy disks, optical disks, Read-Only Memories (ROMs), compact disc ROMs (CD-ROMs), and magnetic-optical disks, Random Access Memories (RAMs), EPROMS, EEPROMs, magnetic or optical cards, or any type of media suitable for storing electronic instructions.

The algorithms and displays presented herein are not inherently related to any particular computer or other apparatus. Various general-purpose systems may be used with programs in accordance with the teachings herein, or it may prove convenient to construct a more specialized apparatus to perform the required method steps. The required structure for a variety of these systems will appear from the description below. In addition, the present embodiments are not described with reference to any particular programming language. It will be appreciated that a variety of programming languages may be used to implement the teachings of the present embodiments as described herein. It should also be noted that the terms “when” or the phrase “in response to,” as used herein, should be understood to indicate that there may be intervening time, intervening events, or both before the identified operation is performed.

It is to be understood that the above description is intended to be illustrative, and not restrictive. Many other embodiments will be apparent to those of skill in the art upon reading and understanding the above description. The scope of the present embodiments should, therefore, be determined with reference to the appended claims, along with the full scope of equivalents to which such claims are entitled.

What is claimed is:

1. An electronic device comprising:
a housing comprising:
a first portion disposed proximate a first end of the electronic device, and
a second portion disposed further from the first end than the first portion;
a first antenna disposed at least partially within the first portion;
a battery disposed at least partially within the first portion;
a wireless communications component disposed at least partially within the first portion;
a second antenna disposed at least partially within the second portion;
a first set of one or more sensors disposed at least partially within the first portion, the first set of one or more sensors comprising a first temperature sensor;
a second set of one or more sensors disposed at least partially within the second portion, the second set of one or more sensors comprising a second temperature sensor;
a first capacitive moisture sensor area disposed at the second portion a first distance from the first portion, and
a second capacitive moisture sensor area disposed at the second portion a second distance from the first portion, the second distance being greater than the first distance.

2. The electronic device of claim 1, wherein, to radiate or receive electromagnetic energy when used by the wireless communications component, the first antenna is a main resonant arm, and the second antenna is a ground element.

3. The electronic device of claim 1, wherein the first antenna is placed above soil and the second antenna is placed inside the soil, wherein the electronic device, to identify whether water is flowing, uses a change in impedance and isolation between the first antenna and the second antenna.

4. A multimodal soil sensor device comprising:
an elongated housing having a first portion and a second portion;
an antenna located in or above the first portion;
a wireless communications component located in the first portion, wherein the wireless communications component is coupled to the antenna, wherein the wireless communications component causes the antenna to radiate or receive electromagnetic energy to communicate with a second device; and
one or more multimodal soil sensors, wherein the one or more multimodal soil sensors is to measure one or more first measurements of a first sensing modality and one or more second measurements of a second sensing modality different than the first sensing modality, and wherein the wireless communications component is to wirelessly send measurement data, comprising the first measurements and the second measurements, to the second device via the antenna.

5. The multimodal soil sensor device of claim 4, wherein the wireless communications component is to communicate the measurement data using the Long Range Wide Area Network (LoRaWAN) protocol or the Bluetooth Low Energy (BLE) protocol.

6. The multimodal soil sensor device of claim 4, wherein the first measurements of the first sensing modality and the second measurements of the second sensing modality are at least two sensing modalities of the following:
temperature measurements;
electrical conductivity (EC) measurements;
Nitrogen-Phosphorus-Potassium (NPK) content measurements;
soil moisture measurements;
a potential of hydrogen (pH) measurements;
relative humidity (Rh) measurements; and
illuminance measurements.

7. The multimodal soil sensor device of claim 4, further comprising:
a first temperature sensor, a first relative humidity (Rh) sensor, and an illuminance sensor located in the first portion;
a second temperature sensor and a first electrical conductivity (EC) sensor located in the second portion at a first distance from a bottom of the elongated housing; and
a third temperature sensor, a second EC sensor, and a potential of hydrogen (pH) sensor located in the second portion at a second distance from the bottom of the elongated housing, the second distance being less than the first distance.

8. The multimodal soil sensor device of claim 7, further comprising:
a first capacitive moisture sensor located in the second portion between the first distance and the first portion; and
a second capacitive moisture sensor located in the second portion between the first distance and the second distance.

9. The multimodal soil sensor device of claim 4, wherein the elongated housing comprises:
a probe housing having first threads at a first end, wherein the probe housing comprises a stem shape to be placed in soil at a second end opposite the first end, wherein the one or more multimodal soil sensors are placed in the probe housing; and
an electronics housing having second threads to mechanically couple with the first threads, wherein the wireless communications component is located within the electronics housing.

10. A soil sensor device comprising:
a housing comprising:
a first portion disposed proximate a first end of the electronic device, and
a second portion disposed further from the first end than the first portion;
a battery disposed at least partially within the housing;
a first antenna disposed at least partially within the first portion;
a wireless communications component disposed at least partially within the first portion;
a second antenna disposed at least partially within the second portion;
a first set of one or more sensors disposed at least partially within the first portion, the first set of one or more sensors comprising a first temperature sensor;
a second set of one or more sensors disposed at least partially within the second portion, the second set of one or more sensors comprising a second temperature sensor;
a first capacitive moisture sensor area disposed at the second portion a first distance from the first portion, and
a second capacitive moisture sensor area disposed at the second portion a second distance from the first portion, the second distance being greater than the first distance.

11. The soil sensor device of claim 10, wherein the soil sensor device comprises one or more computer readable media storing processor executable instructions which, when executed using one or more processors of the soil sensor device, cause the soil sensor device to perform operations comprising
determining, based on a first signal generated using the first antenna and a second signal generated using the second antenna, water flow data indicating whether water is flowing.

12. The soil sensor device of claim 10, wherein the soil sensor device comprises an ambient light sensor disposed at least partially within the first portion.

13. The soil sensor device of claim 10, wherein the soil sensor device comprises a spectroradiometer disposed at least partially within the first portion.

14. The soil sensor device of claim 10, wherein the soil sensor device comprises one or more computer readable media storing processor executable instructions which, when executed using one or more processors of the soil sensor device, cause the soil sensor device to perform operations comprising
transmitting, using the wireless communications component, sensor data using a chirp spread spectrum modulation scheme.

15. The soil sensor device of claim 10, wherein the soil sensor device comprises one or more computer readable media storing processor executable instructions which, when executed using one or more processors of the soil sensor device, cause the soil sensor device to perform operations comprising
transmitting, using the wireless communications component, sensor data using a Bluetooth low energy protocol.

16. The soil sensor device of claim 10, wherein at least part of the second portion operates as at least part of a ground leg for the first antenna.

17. The soil sensor device of claim 10, wherein one or more components at least partially received within the second portion operate as at least part of a ground leg for the first antenna.

18. The soil sensor device of claim 10, wherein the soil sensor device comprises a humidity sensor disposed at least partially within the first portion.

19. The soil sensor device of claim 10, wherein the soil sensor device comprises one or more computer readable media storing processor executable instructions which, when executed using one or more processors of the soil sensor device, cause the soil sensor device to perform operations comprising
determining, using at least one of the second set of one or more sensors, electrical conductivity data.

20. The soil sensor device of claim 10, wherein the soil sensor device comprises one or more computer readable media storing processor executable instructions which, when executed using one or more processors of the soil sensor device, cause the soil sensor device to perform operations comprising
determining, using at least one of the second set of one or more sensors, pH data.
